(19) United States
(12) Patent Application Publication
(10) Pub. No.: US 2008/0046038 A1
(43) Pub. Date: Feb. 21, 2008

(54) LOCAL COMMUNICATIONS NETWORK FOR DISTRIBUTED SENSING AND THERAPY IN BIOMEDICAL APPLICATIONS

(76) Inventors: Gerard J. Hill, Champlin, MN (US); Gregory J. Haubrich, Champlin, MN (US); Sarah A. Audet, Shoreview, MN (US); Richard J. O'Brien, Hugo, MN (US); Javaid Masoud, Shoreview, MN (US)

Correspondence Address: MEDTRONIC, INC., 710 MEDTRONIC PARKWAY NE, MINNEAPOLIS, MN, 55432-9924, US

(21) Appl. No.: 11/739,434

(22) Filed: Apr. 24, 2007

Related U.S. Application Data
(60) Provisional application No. 60/805,789, filed on Jun. 26, 2006.

(51) Int. Cl. A61N 1/02

(52) U.S. Cl. 607/60

(57) ABSTRACT

A local communication network for an implantable medical device system is provided. The system includes a first medical device and a second medical device adapted for implantation in the body of a patient including a telemetry circuit enabled for transmitting data via a wireless communication link to the first medical device. The system further includes a third device comprising signal generating circuitry for generating a wake-up signal. The second implantable medical device transitions from an “off” state to a high-power “on” state in response to the wake-up signal generated by the third device.

### CROSS REFERENCE TO PRIORITY APPLICATION

This application claims priority to application Ser. No. 60/805,789, filed Jun. 26, 2006 and entitled, “Local Communications Network for Distributed Sensing and Therapy in Biomedical Applications”, which is incorporated by reference herein.

### REFERENCE TO RELATED APPLICATIONS

Reference is made to commonly assigned application entitled “COMMUNICATIONS NETWORK FOR DISTRIBUTED SENSING AND THERAPY IN BIOMEDICAL APPLICATIONS”, having docket number P0025563.01 which is filed on even date with the present application and hereby incorporated herein by reference in its entirety.

### TECHNICAL FIELD

The invention relates generally to implantable medical devices and, in particular, to a local communications network for use with implantable sensing and/or therapy delivery devices organized in a distributed network.

### BACKGROUND

A wide variety of implantable medical devices (IMDs) are available for monitoring physiological conditions and/or delivering therapies. Such devices may includes sensors for monitoring physiological signals for diagnostic purposes, monitoring disease progression, or controlling and optimizing therapy delivery. Examples of implantable monitoring devices include hemodynamic monitors, ECG monitors, and glucose monitors. Examples of therapy delivery devices include devices enabled to deliver electrical stimulation pulses such as cardiac pacemakers, implantable cardioverter defibrillators, neurostimulators, and neuromuscular stimulators, and drug delivery devices, such as insulin pumps, morphine pumps, etc.

IMDs are often coupled to medical leads, extending from a housing enclosing the IMD circuitry. The leads carry sensors and/or electrodes and are used to dispose the sensors/electrodes at a targeted monitoring or therapy delivery site while providing electrical connection between the sensor/electrodes and the IMD circuitry. Leadless IMDs have also been described which incorporate electrodes/sensors on or in the housing of the device.

IMD function and overall patient care may be enhanced by including sensors distributed to body locations that are remote from the IMD. However, physical connection of sensors distributed in other body locations to the IMD in order to enable communication of sensed signals to be transferred to the IMD can be cumbersome, highly invasive, or simply not feasible depending on sensor implant location. An acoustic body bus has been disclosed by Funke (U.S. Pat. No. 5,113,859) to allow wireless bidirectional communication through a patient's body. As implantable device technology advances, and the ability to continuously and remotely provide total patient management care expands, there is an apparent need for providing efficient communication between implanted medical devices distributed through a patient's body or regions of a patient's body, as well as with devices.

### BRIEF DESCRIPTION OF THE DRAWINGS

FIG. 1 is a conceptual diagram of a local communication network implemented in an implantable medical device system.

FIG. 2 is a conceptual diagram illustrating a local communication network implemented within a mesh network architecture of an implantable medical device system.

FIG. 3 is a flow chart summarizing communication operations performed by a power-saving, localized network implemented in an implantable medical device system.

FIG. 4 is a functional block diagram of components that may be included in an implantable medical device included in a constellation of distributed devices.

### DETAILED DESCRIPTION

In the following description, references are made to illustrative embodiments for carrying out the invention. It is understood that other embodiments may be utilized without departing from the scope of the invention. For purposes of clarity, the same reference numbers are used in the drawings to identify similar elements. As used herein, the term “module” refers to an application specific integrated circuit (ASIC), an electronic circuit, a processor (shared, dedicated, or group) and memory that execute one or more software or firmware programs, a combinational logic circuit, or other suitable components that provide the described functionality.

The present invention is directed to an ultra-low power, local communications network for use with an implantable medical device system. As used herein, the term “constellation” of devices refers to implantable medical devices deployed to targeted implant sites within signal-receiving range of an implanted or external pinging device. The term “distributed” medical devices refers to implantable devices that are implanted in a distributed manner through the patient's body or a region of the patient's body without being hardwired together by leads or other connectors. Medical devices included in a distributed medical device system will typically include leadless sensors and/or therapy delivery devices positioned at targeted monitoring/therapy delivery sites.

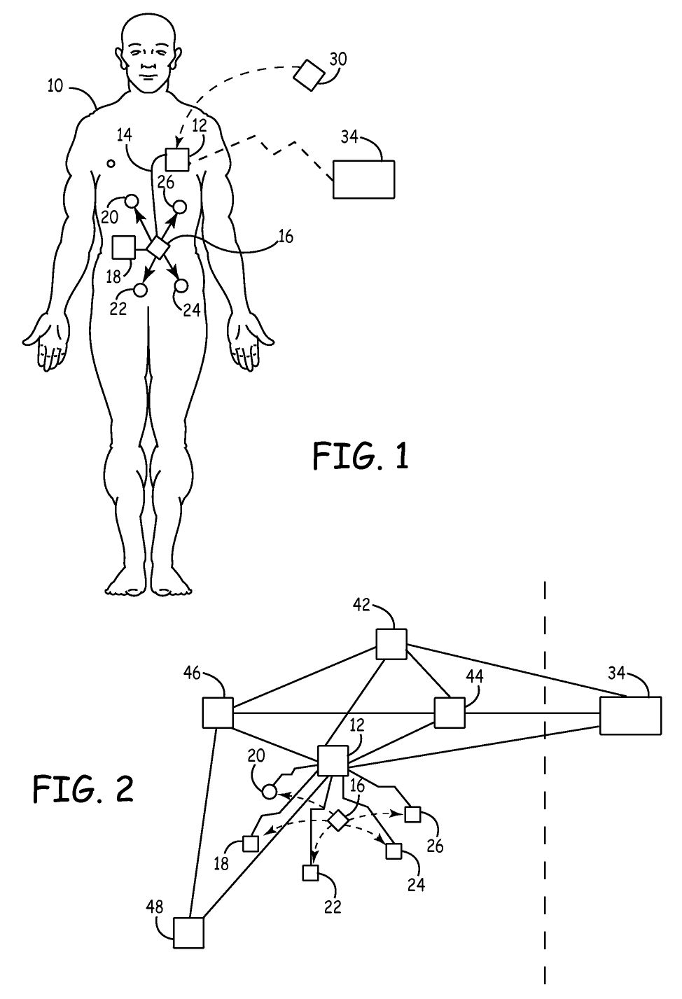

FIG. 1 is a conceptual diagram of a local communication network implemented in an implantable medical device system. An IMD 12 is implanted in a patient 10. IMD 12 is embodied as a cardiac stimulation device capable of delivering cardiac pacing, cardioverting and/or defibrillation therapies as well as sensing cardiac signals and optionally other physiological signals. IMD 12 may alternatively be embodied as any IMD capable of monitoring physiological signals and/or delivering therapy such as a neurostimulator, drug pump, hemodynamic monitor, or ECG monitor.

IMD 12 is shown coupled to a lead 14. Lead 14 carries one or more electrodes for sensing and/or delivering electrical stimulation therapies and may carry additional sensors for monitoring physiological signals. In other embodiments, IMD 12 may be coupled to multiple leads or alternatively be provided as a leadless device, incorporating electrodes and sensors on or in the housing of IMD 12. IMD 12 is enabled for bidirectional communication using RF telemetry or other wireless communication with an external device 34 such as a home monitor or programmer. One example of an appropriate RF telemetry communication system is generally described in commonly-assigned U.S. Pat. No. 6,482,154 (Haubrich, et al.), hereby incorporated herein by reference in its entirety.

Patient 10 is further implanted with a number of other devices 18, 20, 22, 24 and 26 disposed as a constellation of distributed devices. Device 18 may be a second therapy delivery device such as another electrical stimulation device or a drug pump. Devices 20, 22, 24 and 26 are embodied as implantable sensors and may include, but are not limited to, sensors for monitoring pressure, blood flow, acceleration, displacement, or blood/tissue chemistry such as oxygen saturation, carbon dioxide, pH, protein levels, enzyme levels, etc. Devices 12 through 26 represent a distributed system of implantable medical devices in that the devices are not coupled to each other by leads or conductors. Sensors 20 through 26 are implanted at targeted monitoring sites without limitations associated with lead-based sensors.

Devices 12 through 26 are provided with wireless communication connectivity in a local communications network. Devices 18 through 26 are arranged as a “constellation” or cluster of distributed devices within signal reception range of a local network pinging device 16. Local network pinging device 16 is shown coupled to lead 14. In other embodiments, pinging device 16 may also be embodied as a leadless device. Pinging device 16 may alternatively be incorporated in IMD 12 depending on the proximity of IMD 12 to the targeted constellation of devices 18 through 26 for successful receipt of and response to a wake-up signal generated by pinging device 16.

Device 18 and sensors 20 through 26 include a power source, which may be a stand-alone battery, a rechargeable storage device such as a rechargeable battery or capacitor (which may be recharged internally or transcutaneously with the use of electromagnetic or piezoelectric transformers), or an energy-harvesting device. Device 18 and sensors 20 through 26 further include a physiological sensor (which is optional in therapy delivery device 18) and a processor and associated memory for controlling device communication functions and storing data as needed. Device 18 and sensors 20 through 26 are provided with an RF telemetry transmitter or transceiver to allow devices 18 through 26 to transmit data to IMD 12 and/or external device 34.

Device 18 and sensors 20 through 26 are normally in an ultra-low power “OFF,” state and are responsive to an acoustic or RF ping signal generated by pinging device 16. During the OFF state, no active circuitry is consuming power, such that the only energy consumed by the device is due to leakage currents, which are generally in the nA range. No power is consumed by the data communications circuitry, and power control circuitry essentially opens the power supply lines to all power-dependent device circuitry or modules. The power control circuitry is in an OFF state as well.

Pinging device 16 generates a ping signal on a scheduled or manually or automatically triggered basis. The ping signal causes a ping detector included in device 18 and sensors 20 through 26 to wake-up power control circuitry which then wakes up the microprocessor included in device 18 and sensors 20 through 26 thus transitioning device 18 and sensors 20 through 26 to a high power “ON” state. The microprocessor subsequently wakes up communications circuitry. This transition to a high-power “ON” state enables the telemetry circuitry of device 18 and sensors 20 through 26 for receiving commands or requests via an RF communication link in a bidirectional operation mode or for transmitting data in a transmit-only mode. The wake-up response to a ping signal may be based on charge accumulation reaching a wake-up threshold or based on a resonance response to an incident frequency. In one embodiment, the ping detector is an acoustic sensor or transducer which turns on a switch which powers up a bootstrap circuit to take the control and microprocessor circuitry out a an ultra-low power OFF state to a high-power ON state. In an alternative embodiment, the ping detector includes an RF energy detector, e.g., a resonant circuit in RFID or Tag systems) and the energy coupled to the ping detector causes a switch to close subsequently resulting in a powering up of the power control circuitry, microprocessor, communication circuitry and other device components. Other mechanisms for wake-up responses of devices 18 through 26 to a ping signal may be implemented. The response of an acoustic or RF ping detector is rapid allowing minimal latency between generation of a ping signal and initiation of a sensed physiological event. Thus the response time of the overall system can be minimized to allow a rapid response to a changing physiological condition such as the onset of myocardial ischemia and a cardiac arrhythmia.

Upon receiving the wake-up signal from pinging device 16, device 18 and sensors 20 through 26 commence an RF data communication session for transmitting and/or receiving data from IMD 12 and/or an external device 34. Sensors 20 through 26 may be embodied as transmit-only devices for sending data through an RF communication link to IMD 12 or external device 34 using an Aloha supervised communication scheme with redundancy or other communication protocol for reducing data packet collisions. For example, data transmissions may be staggered through time using different time delay signals for each addressed device. If autonomous supervision of data transmission is not implemented, the power consumption of sensors 20 through 26 operating in a transmit-only mode can be extremely low with power being consumed only when a sensor is actively pinged. The longevity of the implanted sensors 20 through 26 may approach the self-discharge rate of the sensor power source.

Sensors 20 through 26 may alternatively be enabled for bi-directional communication and may alternate between transmit-only and bidirectional communication modes depending on the power status of the sensor, the operational workload of the sensor for monitoring physiological signals, and the status of the patient. Device 18 will typically be enabled for bi-directional communication but may also be embodied with transmit-only capabilities.

In past practice, an implanted device is programmed to “wake-up” at prescheduled times or remains in a low-power but “alert” state for receiving communication requests. By providing a pinging device 16 for waking up the devices 18 through 26 from an “OFF” state, communication sessions can be initiated at any time without waiting for a scheduled wake-up of devices 18 through 26. The power consumption burden normally required for maintaining devices 18 through 26 in a low-power “alert” state is reduced by allowing devices 18 through 26 to remain in an even lower power OFF state until actively pinged. By reducing the power required for enabling local communication connectivity in the implanted system, the overall size of the constellation devices 18 through 26 can be reduced.

Pinging device 16 can be implemented as a simple beacon device for waking up all implanted devices 18 through 26. Alternatively, pinging device 16 may be enabled to address individual devices or groups of devices through implementation of an addressing scheme based on frequency, time or digital code.

Device 18 and sensors 20 through 26 may operate in a variety of modes depending on clinician preference and patient condition. For example, device 18 and sensors 20 through 26 may be in an “OFF” state until awoken by pinging device 16 after which the addressed devices are turned “ON” and commence device functions which may include sensing, data processing, therapy delivery, data transmission, or receiving data requests, programming instructions or other data/commands. In other embodiments, device 18 and sensors 20 through 26 may be operating in a low-level state carrying out basic device functions, such as continuous or periodic monitoring of a physiological signal with data storage, and upon receiving a “wake-up” signal from pinging device 16 convert to a high power state for carrying out additional operations such as data processing and data communications. Some devices included in the constellation of distributed devices may be used only at specific times such as during therapy adjustments (e.g., during reprogramming of IMD 12 or during changes in medications or drug dosages). As such, implanted devices 18 through 26 may be available any time a clinician would like to collect additional data or information about the patient's status, remaining in an “OFF” state until actively turned “ON” by pinging device 16.

When pinging device 16 is coupled to IMD 12 by lead 14, pinging device 16 may receive power from conductors extending through lead 14 to the power supply of IMD 12 and receive signals from IMD 12 via conductors extending through lead 14 for triggering pinging device 16 to issue a ping or wake-up signal to one or more of device 18 and sensors 20 through 26. Alternatively, pinging device 16 may be embodied as leadless device having its own power supply (a stand alone battery, rechargeable battery or capacitor, or energy-harvesting device) and enabled for receiving RF telemetry signals from IMD 12 and/or external device 34 for triggering generation of a ping signal. As such, pinging device 16 includes a power supply, a communication link with IMD 12 (which may be wireless or hardwired), and/or a communication link with external device 34 and a signal generator for emitting a ping signal, which may be an acoustical or RF signal, to wake up device 18 and sensors 20 through 26. Pinging device 16 may include a processor and associated memory for controlling the generation of ping signals addressed to specific devices and may operate supervisory protocols for ensuring reliable RF data transmission. Only pinging device 16 need remain in a low-power alert state for receiving communication requests from IMD 12 and/or external device 34, thereby allowing the constellation of distributed devices 18 through 26 to remain in an ultra-low power OFF state.

A local communications network including pinging device 16 may change in membership at any time when new devices are implanted or when existing devices are functionally depleted or physically removed. As such, the constellation of implanted devices can expand “organically” as new sensor and therapy delivery devices are implanted for monitoring and managing a patient's disease progress.

Each of device 18 and sensors 20 through 26 may further be enabled for bidirectional communication with external device 34 to allow for programming of operating modes and control parameters and for transmitting data acquired by the implanted devices 18 through 26 to external device 34. External device 34 may accumulate, prioritize and transfer data as appropriate for notifying the patient 10, a caregiver, a clinician, a clinical database, emergency responders or other external device or communications network of a patient condition, physiological event, or device status. Reference is made to commonly-assigned U.S. Pat. No. 6,599,250 (Webb et al.), U.S. Pat. No. 6,442,433 (Linberg et al.) U.S. Pat. No. 6,622,045 (Snell et al.), U.S. Pat. No. 6,418,346 (Nelson et al.), and U.S. Pat. No. 6,480,745 (Nelson et al.) for general descriptions of examples of network communication systems for use with implantable medical devices for remote patient monitoring and device programming, all of which are hereby incorporated herein by reference in their entirety.

In addition to responding to a ping signal, device 18 and sensors 20 through 26 may be pre-programmed to autonomously wake up and perform sensing, data communication, and other functions at scheduled intervals with data transmitted to IMD 12 and/or external device 34. It is further contemplated that in an awake mode, device 18 and sensors 20 through 26 may communicate with each other in either transmit-only or bidirectional communication modes. RF communication links made available through the implantable medical device system, including both implanted devices and external devices, may be implemented according to the particular application, clinician preference, and individual patient need.

RF communications may be executed between devices 18 through 26 and IMD 12 and/or external device 34 on any selected operating frequency bands such as MICS, MEDS, and ISM. If data from any of the addressed devices is not received by the IMD 12 and/or external device 34 within an expected time window subsequent to generation of the ping signal, the constellation of devices 18 through 26 may be collectively or selectively re-pinged. Repeated attempts may be made according to data priority and communication rules in place, which may be stored in the memory of IMD 12 or pinging device 16.

In some embodiments, a patient may be implanted with a constellation of distributed sensors 20 through 26 for collecting physiological data for diagnostic or patient monitoring purposes without being implanted with a therapy delivery device such as IMD 12. Pinging device 16 operates to wake-up sensors 20 through 26 to initiate data communications and may also receive RF transmitted data from sensors 20 through 26 for storage and transfer to an external device 34. Alternatively or additionally, an external pinging device 30 may be provided which can wake up sensors 20 through 26 to initiate communication operations between sensors 20 through 26 and external device 34. When IMD 12 is present, IMD 12 may also be responsive to an externally generated ping signal from external pinging device 30. External pinging device 30 may be implemented as a stand-alone device that may be manually triggered by a user, such as a patient, caregiver, clinician, or emergency responder. Alternatively, external pinging device 30 may be embodied in external hospital monitoring equipment, an automatic external defibrillator (AED), an external home monitor 34, or a patient activator or other handheld device.

FIG. 2 is a conceptual diagram illustrating a local communication network implemented within a mesh network architecture of an implantable medical device system. IMD 12 may be implemented as a network member (node) of a mesh architecture implantable medical device communication system, as generally described in U.S. patent application Ser. No. ______, (docket number P25563.00). IMD 12 is shown to be networked with multiple implantable devices 42, 44, 46 and 48 and with external device 34. Each of devices 12, 42, 44, 46, 48, and 34 function as nodes of the mesh network allowing multi-hop data transmissions between devices 12, 42, 44, 46, 48, and 34. Each device is enabled to communicate wirelessly along multiple pathways with each of the other networked devices. Only examples of some of the shorter communication pathways are shown in FIG. 2 for the sake of simplicity. The mesh network is a self-configuring, self-healing network responsive to changes in network membership, changes in patient condition, and changes in the individual power status of network members. Implanted networked devices 42, 44, 46 and 48 may include specialized nodes assigned to perform network tasks such as data processing, data storage, gateway, scheduling, etc. Devices 42 through 48 may further include physiological sensing and/or therapy delivery functions.

IMD 12 is configured to receive data packets from the local constellation of device 18 and sensors 20 through 26 responsive to ping signals received from pinging device 16. IMD 12 may then transmit data received from the local constellation of devices 18 through 26 to any of the networked implanted devices 42 through 48 and external device 34 according to a channel plan and routing scheme currently effective in the mesh network. As such data collected by IMD 12 from the local constellation of devices 18 through 26 may be used directly by IMD 12 or transmitted to another device included in the implanted system via the mesh network for use by the other device.

It is contemplated that an individual patient may be implanted with multiple constellations of distributed medical devices, each including a ping device. Each constellation of devices would be disposed within signal-receiving distance from a pinging device for that constellation. When multiple pinging devices are implanted, only one needs to remain in a low-power alert state for receiving a communication request from an IMD or external device. The alert pinging device would then emit a ping signal to “wake-up” the remainder of the pinging devices which would each, in turn, emit pinging signals to their respective constellation of devices. As such each pinging device may also be configured with a processor responsive to a ping signal. The duty of operating as a “wake-up master” could be transferred to different pinging devices based on individual pinging device power status or other patient-related priorities.

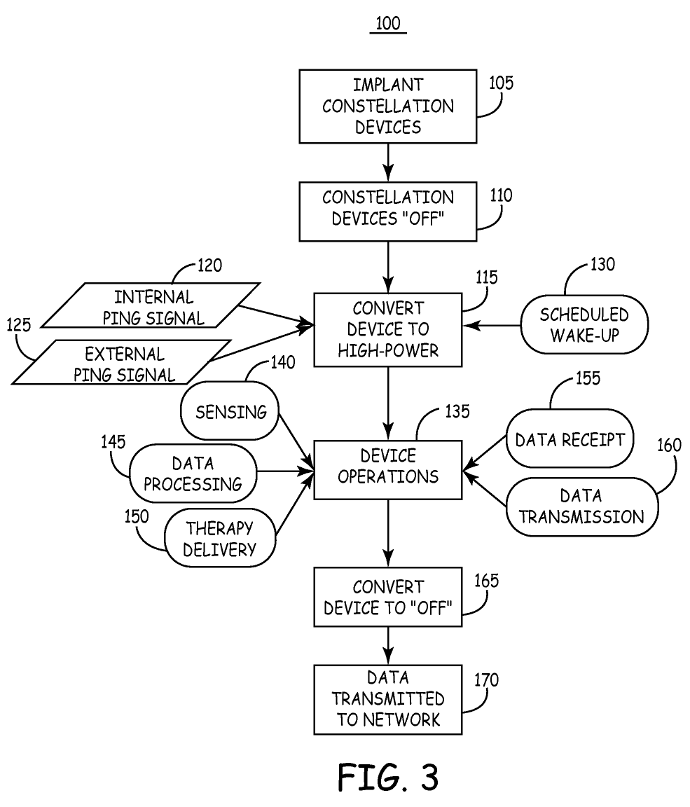

FIG. 3 is a flow chart summarizing communication operations performed by a power-saving localized network implemented in an implantable medical device system. Flow chart 100 is intended to illustrate the functional operation of a local communication system in accordance with one embodiment of the invention, and should not be construed as reflective of a specific form of software or hardware necessary to practice the invention. It is believed that the particular form of software and hardware will be determined primarily by the particular system architecture employed in the implantable and external devices included in the system. Providing software to accomplish the present invention in the context of any modern implantable device system, given the disclosure herein, is within the abilities of one of skill in the art.

At block 105, one or more devices are implanted in a patient forming a “constellation” of distributed medical devices. The implanted devices are normally in an ultra-low power or OFF state as indicated by block 110. The devices transition to a high-power state at block 115 in response to a ping signal (blocks 120 and 125) or a previously scheduled wake-up time. A ping signal may be generated by an implanted device (block 120) or by an external device (block 125) as described previously. One or more devices included in the constellation of devices may be addressed by a ping signal. Upon transitioning to a high-power operating mode, a constellation device will perform device operations according to a received request or a previously programmed operating mode. Device operations may include sensing (block 140), data processing (block 145), therapy deliver (block 150), data receiving (block 155) and/or data transmission (block 160). Typically a device will transmit data to another implanted or external device through RF telemetry communication during the high-power state. At block 165, the device(s) are transitioned back to an “OFF” state upon completion of requested or programmed tasks or upon expiration of a predetermined time interval.

At block 170 data transmitted from a constellation device to an implanted or external device via RF communication link may be further transferred along a communications network such as a mesh communications network of implanted and external devices or to a clinical database or remote patient monitoring system.

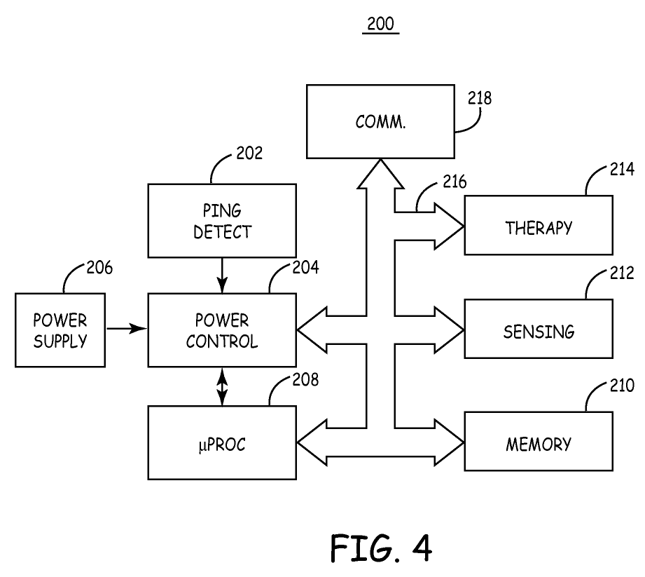

FIG. 4 is a functional block diagram of components that may be included in an implantable medical device included in a constellation of distributed devices. Device 200 includes a ping detector 202 which may be an acoustic transducer or RF resonant circuit, responsive to a ping signal generated by an implanted or external pinging device as described previously. Ping detector 202 wakes up power control module 204 which in turn powers up microprocessor 208. Power control module 204 provides power from power supply 206 to other system components via appropriate power supply lines (not shown). Microprocessor 208 is coupled to other system components such as memory 210, sensing module 212, therapy module 214, and communications module 218 via system bus 216. After microprocessor 201 is transitioned to a high-power state, communications module 218 as well as other functional components such as therapy delivery module 214 and sensing module 212 are activated for performing device functions according to a programmed operating mode or received request under the control of microprocessor 208 and control algorithms stored in memory 210. In various embodiments, a constellation device 200 may include other functional components depending on the type of device and particular application.

Thus, a local communication network for use in an implantable medical device system has been presented in the foregoing description with reference to specific embodiments. It is appreciated that various modifications to the referenced embodiments may be made without departing from the scope of the invention as set forth in the following claims.

1. An implantable medical device system, comprising:
a first medical device;
a second medical device adapted for implantation in the body of a patient comprising a telemetry circuit enabled for transmitting data via a wireless communication link to the first medical device; and
a third device comprising signal generating circuitry for generating a wake-up signal;
wherein the second implantable medical device transitions from an “off” state to a high-power “on” state in response to the wake-up signal generated by the third device.

2. The system of claim 1 wherein the first medical device is an implantable medical device.

3. The system of claim 1 wherein the third device is an implantable medical device.

4. The system of claim 3 wherein the third device is disposed along an implantable medical lead.

5. The system of claim 1 wherein the second medical device comprises a physiological sensor.

6. The system of claim 1 wherein the second medical device comprises a therapy delivery module.

7. The system of claim 1 wherein one of the second medical device and the third medical device comprises one of a battery, a rechargeable energy cell, and an energy-harvesting power source.

8. The system of claim 1 wherein the wake-up signal is one of an acoustic signal and a radio-frequency signal.

9. The system of claim 1 wherein the second device includes a wake-up signal detector.

10. The system of claim 9 wherein the wake-up signal detector is one of an acoustic transducer and an RF energy detector.

11. The system of claim 1 wherein the response of the second device to the wake-up signal comprises a threshold response to a charge accumulation.

12. The system of claim 1 wherein the response of the second device to the wake-up signal comprises a resonance response to an incident frequency of the wake-up signal.

13. The system of claim 1 further comprising a wireless mesh communication network wherein the first medical device is a node of the wireless mesh communication network.

14. The system of claim 1 wherein the second device comprises a plurality of distributed devices and wherein at least one of the plurality of distributed devices operates in a transmit-only mode upon transitioning to the high-power on state.

15. A computer readable medium for storing a set of instructions which, when implemented in an implantable medical device system including a first medical device, a second medical device adapted to be implanted in a patient's body and enabled for telemetric communication with the first medical device, and a third device for generating wake-up signals; cause the system to:
generate a wake-up signal emitted by the third device;
transistion the second medical device from an “off” state to a high-power “on” state in response to the wake-up signal.

16. A method for use in an implantable medical device system including a first medical device, a second medical device adapted to be implanted in a patient's body and enabled for telemetric communication with the first medical device, and a third device for generating wake-up signals, the method comprising:
generating a wake-up signal emitted by the third device;
transistioning the second medical device from an “off” state to a high-power “on” state in response to the wake-up signal.

17. The method of claim 16 wherein generating a wake-up signal comprises generating one of an acoustical signal and an RF signal.

18. The method of claim 16 wherein transitioning the second medical device from an “off” state to a high-power “on” state comprising detecting the wake-up signal by a wake-up signal detector.

19. The method of claim 19 wherein detecting the wake-up signal comprises one of a response to a charge accumulation threshold crossing and an RF resonance response.

20. The method of claim 16 further comprising activating a communication module included in the second medical device.

21. The method of claim 15 further comprising activating a sensing module included in the second medical device.

22. The method of claim 16 further comprising activating a therapy delivery module included in the second medical device.

23. The method of claim 20 further comprising transferring data between the first medical device and the second medical device.

24. The method of claim 20 wherein the first medical device is an implantable medical device.

25. The method of claim 20 further comprising transferring data received by the first medical device from the second medical device to one of an external device or an communications network.

26. The method of claim 25 wherein the communications network is a local mesh communications network.

27. An implantable medical device system, comprising:
a first medical device;
a second medical device adapted to be implanted in a patient's body;
means for providing telemetric communication between the first medical device and the second medical device, and
means for generating a wake-up signal;
means for detecting the wake-up signal wherein the second medical device is transitioned from an off state to a high-power on state in response to detecting the wake-up signal to thereby enable telemetric communication to occur between the first medical device and the second medical device.
